(19) United States
(12) Patent Application Publication
(10) Pub. No.: US 2010/0173293 A1
(43) Pub. Date: Jul. 8, 2010

(54) High Density Sequence Detection Methods

(75) Inventors: Timothy M. Woudenberg, Moss Beach, CA (US); Robert C. Jones, Los Altos, CA (US); Kenneth J. Livak, San Jose, CA (US)

Correspondence Address: LIFE TECHNOLOGIES CORPORATION;C/O INTELLEVATE, P.O. BOX 52050, MINNEAPOLIS, MN, 55402, US

(73) Assignee: Life Technologies Corporation, Carlsbad, CA (US)

(21) Appl. No.: 12/497,398

(22) Filed: Jul. 2, 2009

Related U.S. Application Data
(63) Continuation of application No. 11/828,572, filed on Jul. 26, 2007, now abandoned.
Application No. 11/828,572 is a division of application No. 10/944,686, filed on Sep. 17, 2004, now abandoned.
(60) Provisional application No. 60/504,500, filed on Sep. 19, 2003.
(60) Provisional application No. 60/504,052, filed on Sep. 19, 2003.
(60) Provisional application No. 60/589,224, filed on Jul. 19, 2004.
(60) Provisional application No. 60/589,225, filed on Jul. 19, 2004.
(60) Provisional application No. 60/601,716, filed on Aug. 13, 2004.

(51) Int. Cl. C12Q 1/68; C12P 19/34

(52) U.S. Cl. 435/6; 435/91.2

(57) ABSTRACT

A method for performing PCR on a liquid sample comprising a plurality of polynucleotide targets, wherein each polynucleotide target is present at very low concentration within the sample. The method comprises applying PCR reactants to the surface of a substrate to produce a plurality of reaction spots on the surface of the substrate; loading the liquid sample and a PCR reagent mixture onto the reaction spots; forming a sealed reaction chamber, having a volume of less than about 20 nanoliters, over each of the reaction spots; and amplifying the sample.

### CROSS-REFERENCE TO RELATED APPLICATIONS

This application is a continuation of application Ser. No. 11/828,572 filed Jul. 26, 2007, which is a divisional application of application Ser. No. 10/944,686 filed on Sep. 17, 2004, which claims the benefit of Provisional Application No. 60/504,500 filed on Sep. 19, 2003; Provisional Application No. 60/504,052 filed on Sep. 19, 2003; Provisional Application No. 60/589,224 filed Jul. 19, 2004; Provisional Application No. 60/589,225 filed on Jul. 19, 2004; and Provisional Application No. 60/601,716 filed on Aug. 13, 2004. All of which are incorporated herein by reference.

### INTRODUCTION

The present teachings relate to methods and apparatus for detecting polynucleotides present at very low concentrations in a sample. In particular, such methods relate to methods for detecting the presence of a plurality of nucleotides in a mixture comprising a complex mixture of polynucleotides, using polymerase chain reaction or similar amplifications methods conducted in very small reaction volumes.

Much effort has been dedicated toward mapping of the human genome, which comprises over 3×10⁹ base pairs of DNA (deoxyribonucleic acid). The analysis of the function of the estimated 30,000 human genes is a major focus of basic and applied pharmaceutical research, toward the end of developing diagnostics, medicines and therapies for wide variety of disorders. For example, through understanding of genetic differences between normal and diseased individuals, differences in the biochemical makeup and function of cells and tissues can be determined and appropriate therapeutic interventions identified. However, the complexity of the human genome and the interrelated functions of many genes make the task exceedingly difficult, and require the development of new analytical and diagnostic tools.

A variety of tools and techniques have already been developed to detect and investigate the structure and function of individual genes and the proteins they express. Such tools include polynucleotide probes, which comprise relatively short, defined sequences of nucleic acids, typically labeled with a radioactive or fluorescent moiety to facilitate detection. Probes may be used in a variety of ways to detect the presence of a polynucleotide sequence, to which the probe binds, in a mixture of genetic material. Nucleic acid sequence analysis is also an important tool in investigating the function of individual genes. Several methods for replicating, or “amplifying,” polynucleic acids are known in the art, notably including polymerase chain reaction (PCR). Indeed, PCR has become a major research tool, with applications including cloning, analysis of genetic expression, DNA sequencing, and genetic mapping.

In general, the purpose of a polymerase chain reaction is to manufacture a large volume of DNA which is identical to an initially supplied small volume of “target” or “seed” DNA. The reaction involves copying the strands of the DNA and then using the copies to generate other copies in subsequent cycles. Each cycle will double the amount of DNA present thereby resulting in a geometric progression in the volume of copies of the target DNA strands present in the reaction mixture.

A typical PCR temperature cycle requires that the reaction mixture be held accurately at each incubation temperature for a prescribed time and that the identical cycle or a similar cycle be repeated many times. For example, a PCR program may start at a sample temperature of 94° C. held for 30 seconds to denature the reaction mixture. Then, the temperature of the reaction mixture is lowered to 37° C. and held for one minute to permit primer hybridization. Next, the temperature of the reaction mixture is raised to a temperature in the range from 50° C. to 72° C. where it is held for two minutes to promote the synthesis of extension products. This completes one cycle. The next PCR cycle then starts by raising the temperature of the reaction mixture to 94° C. again for strand separation of the extension products formed in the previous cycle (denaturation). Typically, the cycle is repeated 25 to 30 times.

A variety of devices are commercially available for the analysis of materials using PCR. In order to simultaneously monitor the expression of a large number of genes, high throughput assays have been developed comprising a large number of microarrays of PCR reaction chambers on a microtiter tray or similar substrate. A typical microtiter tray contains 96 or 384 wells on a plate having dimensions of about 72 by 108 mm.

In many situations it would be desirable to test for the presence of multiple target nucleic acid sequences in a starting sample. Such tests would be useful, for example, to detect the presence of multiple different bacteria or viruses in a clinical specimen, to screen for the presence of any of several different sequence variants in microbial nucleic acid associated with resistance to various therapeutic drugs, or to qualitatively and quantitatively analyze the expression of genes in a given biological sample. Such a test would also be useful to screen DNA or RNA from a single individual for sequence variants associated with different mutations in the same or different genes (e.g., single nucleotide polymorphisms, or “SNPs”), or for sequence variants that serve as “markers” for the inheritance of different chromosomal segments from a parent.

However, the ability to perform such analyses on a commercial scale, such as in research laboratories, diagnostic laboratories or the offices of health care providers, presents significant issues, in part because of the vast numbers of polynucleotides to be screened, and the low concentrations in which they are present in biological samples. Such assays must minimize cross contamination between samples, be reproducible, and economical.

### SUMMARY

The present teachings provide methods for amplifying polynucleotides in a liquid sample comprising a plurality of polynucleotide targets, each polynucleotide target being present at very low concentration within the sample, comprising:

(a) applying amplification reactants to the surface of a substrate comprising reaction spots on the surface of the substrate, wherein the amplification reactants comprise the liquid sample and an amplification reagent mixture;

(b) forming a sealed reaction chamber, having a volume of less than about 120 nanoliters, over each of said reaction spots; and

(c) thermal cycling the substrate and reactants.

In some embodiments the amplification is performed by PCR. In some embodiments, the reaction chambers have a volume of less then about 20 nl. In some embodiments, the surface of the substrate comprises a plurality of reaction spots each having a unique probe and set of primers specific for an individual target among said polynucleotide targets. Also, in some embodiments, the applying step comprises the sub-steps of (1) applying said liquid sample to said surface so as to contact said reaction spots; and (2) applying said PCR reagent mixture to said surface so as to contact said reaction spots.

In some embodiments, the forming step comprises loading a sealing fluid, e.g., mineral oil, on said surface of the substrate so as to substantially cover the reaction spots. The present teachings also provide microplates, for use in amplifying polynucleotides in a liquid sample comprising a plurality of polynucleotide targets, comprising:

(a) a substrate having at least about 10,000 reaction spots, each spot comprising a unique PCR primer and a droplet of PCR reagent having a volume of less than about 120 nanoliters, or less then about 20 nanoliters; and

(b) a sealing liquid covering said substrate and isolating each of said reaction spots.

It has been found that the methods and apparatus of the present teachings afford benefits over methods and apparatus among those known in the art. Such benefits include one or more of increased throughput, enhanced accuracy, ability to be used to simultaneously detect and quantify large numbers of polynucleotides, ability to be used with currently available equipment, reduced cost, and enhanced ease of operation. Further benefits and embodiments of the present teachings are apparent from the description set forth herein.

### FIGURES

FIG. 1 depicts an array of the present teachings, comprising a plurality of reaction spots on a planar substrate.

FIG. 2 depicts an embodiment of the present teachings comprising a primer bound to the surface of a substrate.

FIG. 3 depicts an embodiment of the present teachings comprising a primer bound to the surface of a substrate having a hydrogel enhanced attachment surface.

FIG. 4 depicts an embodiment of the present teachings comprising a primer bound to the surface of a substrate having a polymeric enhanced attachment surface.

FIG. 5 depicts a microplate and amplification apparatus useful in the methods of the present teachings.

FIG. 6 depicts the stages in a method of the present teachings.

It should be noted that the figures set forth herein are intended to exemplify the general characteristics of an apparatus, materials and methods among those of the present teachings, for the purpose of the description of such embodiments herein. These figures may not precisely reflect the characteristics of any given embodiment, and are not necessarily intended to define or limit specific embodiments within the scope of the present teachings.

### DESCRIPTION

The present teachings provide methods and apparatus for amplifying polynucleotide targets in a complex mixture of polynucleotides. The following definitions and non-limiting guidelines must be considered in reviewing the description of the present teachings set forth herein.

The headings (such as “Introduction” and “Summary,”) and sub-headings (such as “Amplification”) used herein are intended only for general organization of topics within the disclosure of the present teachings, and are not intended to limit the disclosure of the present teachings or any aspect thereof. In particular, subject matter disclosed in the “Introduction” may include aspects of technology within the scope of the present teachings, and may not constitute a recitation of prior art. Subject matter disclosed in the “Summary” is not an exhaustive or complete disclosure of the entire scope of the present teachings or any embodiments thereof.

The citation of references herein does not constitute an admission that those references are prior art or have any relevance to the patentability of the present teachings. Any discussion of the content of references cited in the Introduction is intended merely to provide a general summary of assertions made by the authors of the references, and does not constitute an admission as to the accuracy of the content of such references. All references cited in the Description section of this specification are hereby incorporated by reference in their entirety.

The description and specific examples, while indicating embodiments of the present teachings, are intended for purposes of illustration only and are not intended to limit the scope of the present teachings. Moreover, recitation of multiple embodiments having stated features is not intended to exclude other embodiments having additional features, or other embodiments incorporating different combinations the stated of features.

As used herein, the words “preferred” and “preferably” refer to embodiments of the present teachings that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the present teachings.

As used herein, the word “include,” and its variants, is intended to be non-limiting, such that recitation of items in a list is not to the exclusion of other like items that may also be useful in the materials, compositions, devices, and methods of the present teachings.

### Amplification

The present teachings provide methods for amplifying polynucleotides. As referred to herein, “polynucleotide” refers to naturally occurring polynucleotides (e.g., DNA or RNA), and analogs thereof, of any length. As referred to herein, the term “amplification” and variants thereof, refer to any process of replicating a “target” polynucleotide (also referred to as a “template”) so as to produce multiple polynucleotides (herein, “amplicons”) that are identical or essentially identical to the target in a sample, thereby effectively increasing the concentration of the target in the sample. In embodiments of the present teachings, amplification of either or both strands of a target polynucleotide comprises the use of one or more nucleic acid-modifying enzymes, such as a DNA polymerase, a ligase, an RNA polymerase, or an RNA-dependent reverse transcriptase. Amplification methods among those useful herein include methods of nucleic acid amplification known in the art, such as Polymerase Chain Reaction (PCR), Ligation Chain Reaction (LCR), Nucleic Acid Sequence Based Amplification (NASBA), self-sustained sequence replication (3SR), strand displacement activation (SDA), Q (3 replicase) system, and combinations thereof. The LCR is, for example, described in the literature, for example, by U. Landegren, et al., “A Ligase-mediated Gene Detection Technique”, Science 241, 1077-1080 (1988). Similarly, NASBA is as described, for example, by J. Cuatelli, et al., “Isothermal in Vitro Amplification of Nucleic Acids by a Multienzyme Reaction Modeled After Retroviral Replication”, Proc. Natl. Acad. Sci. U.S.A 87, 1874-1878 (1990).

In some embodiments, amplification is performed by PCR. As used herein, PCR refers to polymerase chain reaction as well as the reverse-transcription polymerase chain reaction (“RT-PCR”). Polynucleotides that can be amplified include both 2′-deoxyribonucleic acids (DNA) and ribonucleic acids (RNA). When the target to be amplified is an RNA, it may be first reversed-transcribed to yield a cDNA, which can then be amplified in a multiplex fashion. Alternatively, the target RNA may be amplified directly using principles of RT-PCR.

The principles of DNA amplification by PCR and RNA amplification by RT-PCR are well-known in the art, such as are described in the following references, all of which are incorporated by reference herein: U.S. Pat. No. 4,683,195, Mullis et al., issued Jul. 28, 1987; U.S. Pat. No. 4,683,202, Mullis, issued Jul. 28, 1987; U.S. Pat. No. 4,800,159, Mullis et al., issued Jan. 24, 1989; U.S. Pat. No. 4,965,188 Mullis et al., issued Oct. 23, 1990; U.S. Pat. No. 5,338,671 Scalice et al., issued Aug. 16, 1994; U.S. Pat. No. 5,340,728 Grosz et al., issued Aug. 23, 1994; U.S. Pat. No. 5,405,774 Abramson et al., issued Apr. 11, 1995; U.S. Pat. No. 5,436,149 Barnes, issued Jul. 25, 1995; U.S. Pat. No. 5,512,462 Cheng, issued Apr. 30, 1996; U.S. Pat. No. 5,561,058, Gelfand et al., issued Oct. 1, 1996; U.S. Pat. No. 5,618,703 Gelfand et al., issued Apr. 8, 1997; U.S. Pat. No. 5,693,517, Gelfand et al., issued Dec. 2, 1997; U.S. Pat. No. 5,876,978, Willey et al., issued Mar. 2, 1999; U.S. Pat. No. 6,037,129 Cole et al., issued Mar. 14, 2000; U.S. Pat. No. 6,087,098, McKiernan et al., issued Jul. 11, 2000; U.S. Pat. No. 6,300,073 Zhao et al., issued Oct. 9, 2001; U.S. Pat. No. 6,406,891, issued Jun. 18, 2002; U.S. Pat. No. 6,485,917, Yamamoto et al., issued Nov. 26, 2002; U.S. Pat. No. 6,436,677, Gu et al., issued Aug. 20, 2002; Innis et al. In: PCR Protocols A guide to Methods and Applications, Academic Press, San Diego (1990); Schlesser et al. Applied and Environ. Microbiol, 57:553-556 (1991); PCR Technology: Principles and Applications for DNA Amplification (ed. H. A. Erlich, Freeman Press, NY, N.Y., 1992); Mattila et al., Nucleic Acids Res. 19: 4967 (1991); Eckert et al., PCR Methods and Applications 1, 17 (1991), PCR A Practical Approach (eds. McPherson, et al., Oxford University Press, Oxford, 1991); PCR2 A Practical Approach (eds. McPherson, et al., Oxford University Press, Oxford, 1995); PCR Essential Data, J. W. Wiley & Sons, Ed. C. R. Newton, 1995; and PCR Protocols: A Guide to Methods and Applications (Innis, M, Gelfand, D., Sninsky, J. and White, T., eds.), Academic Press, San Diego (1990).

In general, PCR methods comprise the use of at least two primers, a forward primer and a reverse primer, which hybridize to a double-stranded target polynucleotide sequence to be amplified. As referred to herein, a “primer” is a naturally occurring or synthetically produced polynucleotide capable of annealing to a complementary template nucleic acid and serving as a point of initiation for target-directed nucleic acid synthesis, such as PCR or other amplification reaction. Primers may be wholly composed of the standard gene-encoding nucleobases (e.g., cytidine, adenine, guanine, thymine and uracil) or, alternatively, they may include modified nucleobases which form base-pairs with the standard nucleobases and are extendible by polymerases. Modified nucleobases useful herein include 7-deazaguanine and 7-deazaadenine. The primers may include one or more modified interlinkages, such as one or more phosphorothioate or phosphorodithioate interlinkages. In some embodiments, all of the primers used in the amplification methods of the present teachings are DNAn oligonucleotides.

A primer need not reflect the exact sequence of the target but must be sufficiently complementary to hybridize with the target. In some embodiments, the primer is substantially complementary to a strand of the specific target sequence to be amplified. As referred to herein, a “substantially complementary” primer is one that is sufficiently complementary to hybridize with its respective strand of the target to form the desired hybridized product under the temperature and other conditions employed in the amplification reaction. Noncomplementary bases may be incorporated in the primer as long as they do not interfere with hybridization and formation of extension products. In some embodiments, the primers have exact complementarity. In some embodiments, a primer comprises regions of mis-match or non-complementarity with its intended target. As a specific example, a region of non-complementarity maybe included at the 5′-end of a primers, with the remainder of the primer sequence being completely complementary to its target polynucleotide sequence. As another example, non-complementary bases or longer regions of non-complementarity are interspersed throughout the primer, provided that the primer has sufficient complementarity to hybridize to the target polynucleotide sequence under the temperatures and other reaction conditions used for the amplification reaction.

In some embodiments, the primer comprises a double-stranded, labeled nucleic acid region adjacent to a single-stranded region. The single-stranded region comprises a nucleic acid sequence which is capable of hybridizing to the template strand. The double-stranded region, or tail, of the primer can be labeled with a detectable moiety which is capable of producing a detectable signal or which is useful in capturing or immobilizing the amplicon product. In some embodiments, the primer is a single-stranded oligodeoxyribonucleotide. In some embodiments, a primer will include a free hydroxyl group at the 3′ end.

The primer must be sufficiently long to prime the synthesis of extension products in the presence of the polymerization agent, depending on such factors as the use contemplated, the complexity of the target sequence, reaction temperature and the source of the primer. Generally, each primer used in the present teachings will have from about 12 to about 40 nucleotides, from about 15 to about 40, or from about 20 to about 40 nucleotides, or from about 20 to about 35 nucleotides. In some embodiments, the primer comprises from about 20 to about 25 nucleotides. Short primer molecules generally require lower temperatures to form sufficiently stable hybrid complexes with the template.

In some embodiments, the amplification primers are designed to have a melting temperature (“Tm”) in the range of about 60-75° C. Melting temperatures in this range will tend to insure that the primers remain annealed or hybridized to the target polynucleotide at the initiation of primer extension. The actual temperature used for the primer extension reaction may depend upon, among other factors, the concentration of the primers which are used in the multiplex assays. For amplifications carried out with a thermostable polymerase such as Taq DNA polymerase, the amplification primers can be designed to have a Tm in the range of from about 60 to about 78° C. In some embodiments, the melting temperatures of different amplification primers used in the same amplification reaction are different. In some embodiments, the melting temperatures of the different amplification primers are approximately the same.

In some embodiments, primers are used in pairs of forward and reverse primers, referred to herein as a “primer pair.” The amplification primer pairs may be sequence-specific and may be designed to hybridize to sequences that flank a sequence of interest to be amplified. Primer pairs can comprise a set of primers including a 5′ upstream primer that hybridizes with the 5′ end of the target sequence to be amplified and a 3′, downstream primer that hybridizes with the complement of the 3′ end of the target sequence to be amplified. Methods useful herein for designing primer pairs suitable for amplifying specific sequences of interest include methods that are well-known in the art.

In PCR, a double-stranded target DNA polynucleotide which includes the sequence to be amplified is incubated in the presence of a primer pair, a DNA polymerase and a mixture of 2′-deoxyribonucleotide triphosphates (“dNTPs”) suitable for DNA synthesis. A variety of different DNA polymerases are useful in the methods of the present teachings. In some embodiments, the polymerase is a thermostable polymerase. Suitable thermostable polymerases include Taq and Tth polymerases, commercially available from Applied Biosystems, Inc., Foster City, Calif., U.S.A.

To begin the amplification, the double-stranded target DNA polynucleotide is denatured and one primer is annealed to each strand of the denatured target. The primers anneal to the target DNA polynucleotide at sites removed from one another and in orientations such that the extension product of one primer, when separated from its complement, can hybridize to the other primer. Once a given primer hybridizes to the target DNA polynucleotide sequence, the primer is extended by the action of the DNA polymerase. The extension product is then denatured from the target sequence, and the process is repeated.

In successive cycles of this process, the extension products produced in earlier cycles serve as templates for subsequent DNA synthesis. Beginning in the second cycle, the product of the amplification begins to accumulate at a logarithmic rate. The final amplification product, or amplicon, is a discrete double-stranded DNA molecule consisting of: (i) a first strand which includes the sequence of the first primer, which is followed by the sequence of interest, which is followed by a sequence complementary to that of the second primer and (ii) a second strand which is complementary to the first strand.

In embodiments for amplifying an RNA target, RT-PCR a single-stranded RNA target which includes the sequence to be amplified (e.g, an mRNA) is incubated in the presence of a reverse transcriptase, two amplification primers, a DNA polymerase and a mixture of dNTPs suitable for DNA synthesis. One of the amplification primers anneals to the RNA target and is extended by the action of the reverse transcriptase, yielding an RNA/cDNA doubled-stranded hybrid. This hybrid is then denatured, and the other primer anneals to the denatured cDNA strand. Once hybridized, the primer is extended by the action of the DNA polymerase, yielding a double-stranded cDNA, which then serves as the double-stranded template or target for further amplification through conventional PCR, as described above. Following reverse transcription, the RNA can remain in the reaction mixture during subsequent PCR amplification, or it can be optionally degraded by well-known methods prior to subsequent PCR amplification. RT-PCR amplification reactions may be carried out with a variety of different reverse transcriptases, although in some embodiments thermostable reverse-transcriptions are preferred. Suitable thermostable reverse transcriptases include, but are not limited to, reverse transcriptases such as AMV reverse transcriptase, MuLV, and Tth reverse transcriptase.

Temperatures suitable for carrying out the various denaturation, annealing and primer extension reactions with the polymerases and reverse transcriptases are well-known in the art. Optional reagents commonly employed in conventional PCR and RT-PCR amplification reactions, such as reagents designed to enhance PCR, modify Tm, or reduce primer-dimer formation, may also be employed in the multiplex amplification reactions. Such reagents are described in U.S. Pat. No. 6,410,231, Arnold et al., issued Jun. 25, 2002; U.S. Pat. No. 6,482,588, Van Doorn et al., issued Nov. 19, 2002; U.S. Pat. No. 6,485,903, Mayrand, issued Nov. 26, 2002; and U.S. Pat. No. 6,485,944, Church et al., issued Nov. 26, 2002. In some embodiments, the multiplex amplifications may be carried out with commercially-available amplification reagents, such as, for example, AmpliTaq® Gold PCR Master Mix, TaqMan® Universal Master Mix and TaqMan® Universal Master Mix No AmpErase® UNG, all of which are available commercially from Applied Biosystems (Foster City, Calif., U.S.A.).

In some embodiments, the amplification reaction is conducted under conditions allowing for quantitative and qualitative analysis of one or more polynucleotide targets. Accordingly, some methods of the present teachings comprise the use of detection reagents, for detecting the presence of a target amplicon in a amplification reaction mixture. In some embodiments, the detection reagent comprises a probe or system of probes having physical (e.g., fluorescent) or chemical properties that change upon hybridization of the probe to a nucleic acid target. As used herein, the term “probe” refers to a polynucleotide of any suitable length which allows specific hybridization to a polynucleotide, e.g., a target or amplicon.

Oligonucleotide probes may be DNA, RNA, PNA, LNA or chimeras comprising one or more combinations thereof. The oligonucleotides may comprise standard or non-standard nucleobases or combinations thereof, and may include one or more modified interlinkages. The oligonucleotide probes may be suitable for a variety of purposes, such as, for example to monitor the amount of an amplicon produced, to detect single nucleotide polymorphisms, or other applications as are well-known in the art. Probes may be attached to a label or reporter molecule. Any suitable method for labeling nucleic acid sequences can be used, e.g., fluorescent labeling, biotin labeling or enzyme labeling.

In some embodiments, an oligonucleotide probe is complementary to at least a region of a specified amplicon. The probe can be completely complementary to the region of the specified amplicons, or may be substantially complementary thereto. In some embodiments, the probe is at least about 65% complementary over a stretch of at least about 15 to about 75 nucleotides. In other embodiments, the probes are at least about 75%, 85%, 90%, or 95% complementary to the regions of the amplicons. Such probes are disclosed, for example, in Kanehisa, M., 1984, Nucleic Acids Res. 12: 203. The exact degree of complementarity between a specified oligonucleotide probe and amplicon will depend upon the desired application for the probe and will be apparent to those of skill in the art.

The length of a probes can vary broadly, and in some embodiments can range from a few as two as many as tens or hundreds of nucleotides, depending upon the particular application for which the probe was designed. In some embodiments, the probe ranges in length from about 15 to about 35 nucleotides. In some embodiments, the oligonucleotide probe ranges in length from about 15 to about 25 nucleotides. In some embodiments, the probe is a “tailed” oligonucleotide probe ranging in length from about 25 to about 75 nucleotides.

In some embodiments of quantitative or real-time amplification assays useful herein, total RNA from a sample is amplified by RT-PCR in the presence of amplification primers suitable for specifically amplifying a specified gene sequence of interest and an oligonucleotide probe labeled with a labeling system that permits monitoring of the quantity of amplicon that accumulates in the amplification reaction in real-time. The cycle threshold values (Ct values) obtained in such quantitative RT-PCR amplification reactions can be correlated with the number of gene copies present in the original total mRNA sample. Such quantitative or real-time RT-PCR reactions, as well as different types of labeled oligonucleotide probes useful for monitoring the amplification in real time, are well-known in the art. Oligonucleotide probes suitable for monitoring the amount of amplicon(s) produced as a function of time, include the 5′-exonuclease assay (TaqMan®) probes; various stem-loop molecular beacons; stemless or linear beacons; peptide nucleic acid (PNA) molecular beacons; linear PNA beacons; non-FRET probes; sunrise primers; scorpion probes; cyclicons; PNA light-up probes; self-assembled nanoparticle probes, and ferrocene-modified probes. Such probes are described in U.S. Pat. No. 6,103,476, Tyagi et al., issued Aug. 15, 2000; U.S. Pat. No. 5,925,517, Tyagi et al., issued Jul. 20, 1999; Tyagi & Kramer, 1996, Nature Biotechnology 14:303-308; PCT Publication No. WO 99/21881, Gildea et al., published May 6, 1999; U.S. Pat. No. 6,355,421, Coull et al., issued Mar. 12, 2002; Kubista et al, 2001, SPIE 4264:53-58; U.S. Pat. No. 6,150,097, Tyagi et al., issued Nov. 21, 2000; U.S. Pat. No. 6,485,901, Gildea et al., issued Nov. 26, 2002; Mhlanga, et al., (2001) Methods. 25:463-471; Whitcombe et al. (1999) Nat Biotechnol. 17:804-807; Isacsson et al. (2000) Mol Cell Probes. 14: 321-328: Svanvik et al (2000) Anal Biochent 281:26-35; Wolff et. al. (2001) Biotechniques 766:769-771; Tsourkas et al (2002) Nucleic Acids Res. 30:4208-4215; Riccelli, et al. (2002) Nucleic Acids Res. 30:4088-4093; Zhang et al. (2002) Shanghai 34:329-332; Maxwell et al. (2002) J. Am Chem Soc. 124:9606-9612; Broude et al. (2002) Trends Biotechnol 20:249-56; Huang et al. (2002) Chem Res Toxicol. 15:118-126; and Yn et al. (2001) J. Am. Chem. Soc. 14: 11155-11161.

In some embodiments, the oligonucleotide probes are suitable for detecting single nucleotide polymorphisms, as is well-known in the art. A specific example of such probes includes a set of four oligonucleotide probes which are identical in sequence save for one nucleotide position. Each of the four probes includes a different nucleotide (A, G, C and T/U) at this position. The probes may be labeled with labels capable of producing different detectable signals that are distinguishable from one another, such as different fluorophores capable of emitting light at different, spectrally-resolvable wavelengths (e.g., 4-differently colored fluorophores). Such labeled probes are known in the art and described, for example, in U.S. Pat. No. 6,140,054, Wittwer et al., issued Oct. 31, 2000; and Saiki et al., 1986, Nature 324:163-166.

One embodiment, which utilizes the 5′-exonuclease assay to monitor the amplification as a function of time is referred to as the 5′-exonuclease gene quantification assay. Such assays are disclosed in U.S. Pat. No. 5,210,015, Gelfand et al., issued May 11, 1993; U.S. Pat. No. 5,538,848, Livak et al., issued Jul. 23, 1996; and Lie & Petropoulos, 1998, Curr. Opin. Biotechnol. 14:303-308).

In specific embodiments, the level of amplification can be determined using a fluorescently labeled oligonucleotide, such as disclosed in Lee, L. G., et al. Nucl. Acids Res. 21:3761 (1993), and Livak, K. J., et al. PCR Methods and Applications 4:357 (1995). In such embodiments, the detection reagents include a sequence-selective primer pair as in the more general PCR method above, and in addition, a sequence-selective oligonucleotide (FQ-oligo) containing a fluorescer-quencher pair. The primers in the primer pair are complementary to 3′-regions in opposing strands of the target segment which flank the region which is to be amplified. The FQ-oligo is selected to be capable of hybridizing selectively to the analyte segment in a region downstream of one of the primers and is located within the region to be amplified.

The fluorescer-quencher pair includes a fluorescer dye and a quencher dye which are spaced from each other on the oligonucleotide so that the quencher dye is able to significantly quench light emitted by the fluorescer at a selected wavelength, while the quencher and fluorescer are both bound to the oligonucleotide. The FQ-oligo can include a 3′-phosphate or other blocking group to prevent terminal extension of the 3′-end of the oligo. The fluorescer and quencher dyes may be selected from any dye combination having the proper overlap of emission (for the fluorescer) and absorptive (for the quencher) wavelengths while also permitting enzymatic cleavage of the FQ-oligo by the polymerase when the oligo is hybridized to the target. Suitable dyes, such as rhodamine and fluorescein derivatives, and methods of attaching them, are well known and are described, for example, in, U.S. Pat. No. 5,188,934, Menchen, et al., issued Feb. 23, 1993, 1993; PCT Publication No. WO 94/05688, Menchen, et al., published Mar. 17, 1994). PCT Publication No. WO 91/05060, Bergot, et al., published Apr. 18, 1991; and European Patent Publication 233,053, Fung, et al., published Aug. 19, 1987. The fluorescer and quencher dyes are spaced close enough together to ensure adequate quenching of the fluorescer, while also being far enough apart to ensure that the polymerase is able to cleave the FQ-oligo at a site between the fluorescer and quencher. Generally, spacing of about 5 to about 30 bases is suitable, as described in Livak, K. J., et al. PCR Methods and Applications 4:357 (1995). In some embodiments, the fluorescer in the FQ-oligo is covalently linked to a nucleotide base which is 5′ with respect to the quencher.

In practicing this approach, the primer pair and FQ-oligo are reacted with a target polynucleotide (double-stranded for this example) under conditions effective to allow sequence-selective hybridization to the appropriate complementary regions in the target. The primers are effective to initiate extension of the primers via DNA polymerase activity. When the polymerase encounters the FQ-probe downstream of the corresponding primer, the polymerase cleaves the FQ-probe so that the fluorescer is no longer held in proximity to the quencher. The fluorescence signal from the released fluorescer therefore increases, indicating that the target sequence is present. In a further embodiment, the detection reagents may include two or more FQ-oligos having distinguishable fluorescer dyes attached, and which are complementary for different-sequence regions which may be present in the amplified region, e.g., due to heterozygosity. See, Lee, L. G., et al. Nucl. Acids Res. 21:3761 (1993)

In some embodiments, the detection reagents include first and second oligonucleotides effective to bind selectively to adjacent, contiguous regions of a target sequence in the selected analyte, and which may be ligated covalently by a ligase enzyme or by chemical means Such oligonucleotide ligation assays (OLA) are as described in U.S. Pat. No. 4,883,750, Whiteley, et al., issued Nov. 28, 1989; and Landegren, U., et al., Science 241:1077 (1988). In this approach, the two oligonucleotides (oligos) are reacted with the target polynucleotide under conditions effective to ensure specific hybridization of the oligonucleotides to their target sequences. When the oligonucleotides have base-paired with their target sequences, such that confronting end subunits in the oligos are base paired with immediately contiguous bases in the target, the two oligos can be joined by ligation, e.g., by treatment with ligase. After the ligation step, the detection wells are heated to dissociate unligated probes, and the presence of ligated, target-bound probe is detected by reaction with an intercalating dye or by other means. The oligos for OLA may also be designed so as to bring together a fluorescer-quencher pair, as discussed above, leading to a decrease in a fluorescence signal when the analyte sequence is present.

In the above OLA ligation method, the concentration of a target region from an analyte polynucleotide can be increased, if necessary, by amplification with repeated hybridization and ligation steps. Simple additive amplification can be achieved using the analyte polynucleotide as a target and repeating denaturation, annealing, and ligation steps until a desired concentration of the ligated product is achieved.

Alternatively, the ligated product formed by hybridization and ligation can be amplified by ligase chain reaction (LCR), according to published methods. See, Winn-Deen, E., et al., Clin. Chem. 37:1522 (1991). In this approach, two sets of sequence-specific oligos are employed for each target region of a double-stranded nucleic acid. One probe set includes first and second oligonucleotides designed for sequence-specific binding to adjacent, contiguous regions of a target sequence in a first strand in the target. The second pair of oligonucleotides are effective to bind (hybridize) to adjacent, contiguous regions of the target sequence on the opposite strand in the target. With continued cycles of denaturation, reannealing and ligation in the presence of the two complementary oligo sets, the target sequence is amplified exponentially, allowing small amounts of target to be detected and/or amplified. In a further modification, the oligos for OLA or LCR assay bind to adjacent regions in a target polynucleotide which are separated by one or more intervening bases, and ligation is effected by reaction with (i) a DNA polymerase, to fill in the intervening single stranded region with complementary nucleotides, and (ii) a ligase enzyme to covalently link the resultant bound oligonucleotides. See, e.g., PCT Publication No. WO 90/01069, Segev, issued Feb. 8, 1990, and Segev, D., “Amplification of Nucleic Acid Sequences by the Repair Chain Reaction” in Nonradioactive Labeling and detection of Biomolecules, C. Kessler (Ed.), Springer Laboratory, Germany (1992).

In some embodiments, the target sequences can be detected on the basis of a hybridization-fluorescence assay. See, e.g., Lee, L. G., et al. Nucl. Acids Res. 21:3761 (1993). The detection reagents include a sequence-selective binding polymer (FQ-oligo) containing a fluorescer-quencher pair, as discussed above, in which the fluorescence emission of the fluorescer dye is substantially quenched by the quencher when the FQ-oligo is free in solution (i.e., not hybridized to a complementary sequence). Hybridization of the FQ-oligo to a complementary sequence in the target to form a double-stranded complex is effective to perturb (e.g., increase) the fluorescence signal of the fluorescer, indicating that the target is present in the sample. In some embodiments, the binding polymer contains only a fluorescer dye (but not a quencher dye) whose fluorescence signal either decreases or increases upon hybridization to the target, to produce a detectable signal.

In some embodiments, the amplified sequences may be detected in double-stranded form by including an intercalating or crosslinking dye, such as ethidium bromide, acridine orange, or an oxazole derivative, for example, which exhibits a fluorescence increase or decrease upon binding to double-stranded nucleic acids. Such methods are described, for example, in Sambrook, J., et al., Molecular Cloning, 2nd Ed., Cold Spring Harbor Laboratory Press, N.Y. (1989); Ausubel, F. M., et al., Current Protocols in Molecular Biology, John Wiley & Sons, Inc., Media, Pa.; Higuchi, R., et al., Bio/Technology 10:413 (1992); Higuchi, R., et al., Bio/Technology 11:1026 (1993); and Ishiguro, T., et al., Anal. Biochem. 229:207 (1995). In a specific embodiment the dye is SYBR® Green I or II, marketed by Molecular Probes (Eugene, Oreg., U.S.A.).

### Materials, Compositions and Devices

The present teachings provide microplates, for use in amplifying polynucleotides in a liquid sample comprising a plurality of polynucleotide targets. In embodiments of the present teachings, such microplates comprise a substrate and a plurality of reaction spots.

### Substrate:

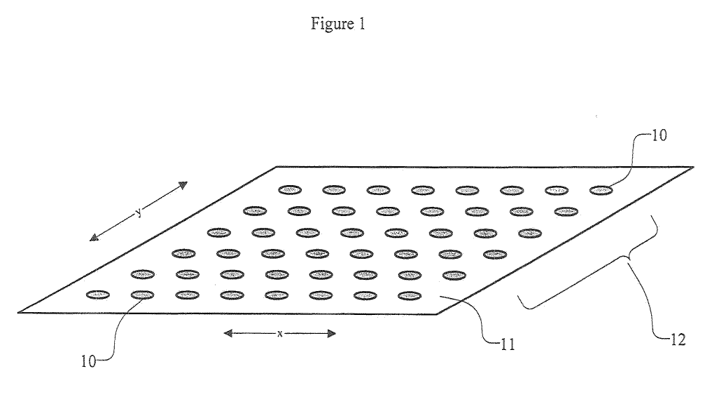

Methods of the present teachings comprise applying PCR reactants to the surface of a substrate, wherein the substrate comprises reaction spots on the surface of the substrate. As referred to herein, a “substrate” is a material comprising a surface which is suitable for support and/or containment of reactants for amplifying polynucleotides according to methods of the present teachings. In some embodiments, the substrate is substantially planar, having substantially planar upper and lower surfaces, wherein the dimensions of the planar surfaces in the x- and y-dimensions are substantially greater than the thickness of the substrate in the z-direction. An embodiment of such a substrate is depicted in FIG. 1, wherein a plurality of reaction spots (10) are formed on the surface (11) of a substantially planar substrate (12).

In some embodiments, the substrate is a plate having dimensions such that the substrate may be used in conventional PCR equipment. In some embodiments, the substrate is from about 50 to about 200 mm in width, and from about 50 to about 200 mm in length. In some embodiments, the substrate is from about 50 to about 100 mm in width, and from about 100 to about 150 mm in length. In some embodiments, the substrate is about 72 mm wide and about 108 mm long.

The substrate may be made of any material which is suitable for conducting amplification of polynucleotides, such as by PCR. In some embodiments, the material is substantially non-reactive with polynucleotides and reagents employed in the amplification reactions with which it is to be used. In some embodiments the material does not interfere with imaging of the amplification reaction (as discussed herein). In embodiments in which imaging is performed by detection of fluorescent labeled reagents, the material may be opaque to transmission of light emitted by the fluorescent labeled reagents. The material can be suitable for use in the manufacturing methods by which reaction spots are formed (as discussed herein).

Substrate materials useful herein include those comprising glass, silicon, quartz, nylon, polystyrene, polyethylene, polypropylene, polytetrafluoroethylene, metal, and combinations thereof. In some embodiments, the substrate comprises glass. In some embodiments, the substrate comprises plastic, such as polycarbonate.

### Reaction Spots:

As referred to herein, a “reaction spot” is a defined area on a substrate which localizes reagents required for amplification of a polynucleotide in sufficient quantity, proximity, and isolation from adjacent areas on the substrate (such as other reaction spots on the substrate), so as to facilitate amplification of one or more polynucleotides in the reaction spot. Such localization is accomplished by physical and chemical modalities, including physical containment of reagents in one dimension and chemical containment in one or more other dimensions. Such physical containment is effected by the surface of the substrate itself, such that the surface forms the bottom of the reaction spot. (As used herein, such terms as “top” and “bottom” are descriptive of orientation of parts or aspects of devices or materials relative to one another, and are not intended to define the absolute orientation of such devices, materials or aspects thereof relative to the user or the earth.) Containment of the reaction spot in other dimensions is effected primarily through chemical modalities, such as through the chemical characteristics of the surface of the substrate surrounding the spot, containment fluids, binding of one or more reagents to the surface, and combinations thereof. Such localization of reagents is contrasted to containment of reagents in wells, wherein reagents are contained through primarily physical means in three or more dimensions (e.g, the bottom and sides of the well).

In some embodiments, the reaction spot comprises an amplification reagent, wherein the amplification reagent is affixed or otherwise contained on or in the reaction spot in such a manner so as to be available for reaction in an amplification method of the present teachings. As referred to here, an “amplification reagent” is a reagent which is used in an amplification reaction of the present teachings, e.g., PCR. In some embodiments, the amplification reagent comprises a primer. In some embodiments, the amplification reagent comprises a primer pair.

In some embodiments, the reaction spot comprises a detection reagent, comprising a reagent which is affixed or otherwise contained on or in the reaction spot in such a manner so as to be available for hybridization to a polynucleotide of interest. In some embodiments, the amplification reagent comprises a probe. In some embodiments, the reaction spot comprises a primer pair for a specific target, and probe for that target.

In some embodiments, the surface of the array comprises an “enhanced reaction surface” which comprises a physical or chemical modification of the surface of the substrate so as to enhance support of an amplification reaction. Such modifications may include chemical treatment of the surface, or coating the surface. In embodiments of the present teachings, such chemical treatment comprises chemical treatment or modification of the surface of the array so as to form hydrophilic and hydrophobic areas. In a certain embodiments, an array (herein, a “surface tension array”) is formed comprising a pattern, such as a regular pattern, of hydrophilic and hydrophobic areas. In some embodiments, a surface tension array comprises a plurality of hydrophilic sites, forming reaction spots, against a hydrophobic matrix, the hydrophilic sites are spatially segregated by hydrophobic regions. Reagents delivered to the array are constrained by surface tension difference between hydrophilic and hydrophobic sites.

In some embodiments, hydrophobic sites may be formed on the surface of the substrate by forming the surface, or chemically treating it, with compounds comprising alkyl groups. In some embodiments, hydrophilic sites may be formed on the surface of the substrate by forming the surface, or chemically treating it, with compounds comprising free amino, hydroxyl, carboxyl, thiol, amido, halo, or sulfate groups. In some embodiments, the free amino, hydroxyl, carboxyl, thiol, amido, halo, or sulfate group of the hydrophilic sites is covalently coupled with a linker moiety (e.g., polylysine, hexethylene glycol, and polyethylene glycol). A variety of methods of forming surface tension arrays useful herein are known in the art. Such methods are described in U.S. Pat. No. 5,985,551, Brennan, issued Nov. 16, 1999; and U.S. Pat. No. 5,474,796, Brennan, issued Dec. 12, 1995.

In some embodiments, surface tension arrays are formed by photoresist methods, including such methods as are known in the art. In some embodiments, a surface tension array is formed by coating a substrate with a photoresist substance and then using a generic photomask to define array patterns on the substrate by exposing them to light. The exposed surface is then reacted with a suitable reagent to form a stable hydrophobic matrix. Such reagents include fluoroalkylsilane or long chain alkylsilane, such as octadecylsilane. The remaining photoresist substance is then removed and the solid support reacted with a suitable reagent, such as aminoalkyl silane or hydroxyalkyl silane, to form hydrophilic regions.

In some embodiments, the substrate is first reacted with a suitable derivatizing reagent to form a hydrophobic surface. Such reagents include vapor or liquid treatment of fluoroalkylsiloxane or alkylsilane. The hydrophobic surface may then be coated with a photoresist substance, photopatterned and developed.

In some embodiments, the exposed hydrophobic surface is reacted with suitable derivatizing reagents to form hydrophilic sites. For example, the exposed hydrophobic surface may be removed by wet or dry etch such as oxygen plasma and then derivatized by aminoalkylsilane or hydroxylalkylsilane treatment. The photoresist coat is then removed to expose the underlying hydrophobic sites.

In some embodiments, the substrate is first reacted with a suitable derivatizing reagent to form a hydrophilic surface. Suitable reagents include vapor or liquid treatment of aminoalkylsilane or hydroxylalkylsilane. The derivatized surface is then coated with a photoresist substance, photopatterned, and developed. The exposed surface may be reacted with suitable derivatizing reagents to form hydrophobic sites. For example, the hydrophobic sites may be formed by fluoroalkylsiloxane or alkylsilane treatment. The photoresist coat may be removed to expose the underlying hydrophilic sites.

A variety of photoresist substances and treatments useful herein are known in the art. Such treatments include optical positive photoresist substances (e.g., AZ 1350, Novolac, marketed by Hoechst Celanese) and E-beam positive photoresist substances (e.g., EB-9™, polymethacrylate, marketed by Hoya Corporation, San Jose, Calif., U.S.A).

A variety of hydrophilic and hydrophobic derivatizing reagents useful herein are also well known in the art. In some embodiments, fluoroalkylsilane or alkylsilane may be employed to form a hydrophobic surface and aminoalkyl silane or hydroxyalkyl silane may be used to form hydrophilic sites. Siloxane derivatizing reagents include those selected from the group consisting of: hydroxyalkyl siloxanes, such as allyl trichlorochlorosilane, and 7-oct-l-enyl trichlorochlorosilane; diol(bis-hydroxyalkyl)siloxanes; glycidyl trimethoxysilanes; aminoalkyl siloxanes, such as 3-aminopropyl trimethoxysilane; Dimeric secondary aminoalkyl siloxanes, such as bis(3-trimethoxysilylpropyl)amine; and combinations thereof.

In some embodiments, a substrate for use in surface tension array comprises glass. Such arrays using a glass substrate may be patterned using numerous techniques developed by the semiconductor industry using thick films (from about 1 to about 5 microns) of photoresists to generate masked patterns of exposed surfaces. After sufficient cleaning, such as by treatment with O₂ radical (e.g., using an O₂ plasma etch, ozone plasma treatment) followed by acid wash, the glass surface may be derivatized with a suitable reagent to form a hydrophilic surface. In some embodiments, the glass surface may be uniformly aminosilylated with an aminosilane, such as aminobutyldimethylmethoxysilane (DMABS). The derivatized surface is then coated with a photoresist substance, soft-baked, photopatterned using a generic photomask to define the array patterns by exposing them to light, and developed. The underlying hydrophilic sites are thus exposed in the mask area and ready to be derivatized again to form hydrophobic sites, while the photoresist covering region protects the underlying hydrophilic sites from further derivatization. Suitable reagents, such as fluoroalkylsilane or long chain alkylsilane, may be employed to form hydrophobic sites. For example, the exposed hydrophilic sites may be burned out with an O₂ plasma etch. The exposed regions may then be fluorosilylated. Following the hydrophobic derivatization, the remaining photoresist can be removed, for example by dissolution in warm organic solvents such as methyl isobutyl ketone or N-methylpyrrolidone (NMP), to expose the hydrophilic sites of the glass surface. For example, the remaining photoresist may be dissolved off with sonication in acetone and then washed off in hot NMP.

In some embodiments, surface tension arrays are made without the use of photoresist. In some embodiments, a substrate is first reacted with a reagent to form hydrophilic sites. Certain of the hydrophilic sites are protected with a suitable protecting agent. The remaining, unprotected, hydrophilic sites are reacted with a reagent to form hydrophobic sites. The protected hydrophilic sites are then deprotected. In some embodiments, a glass surface may be first reacted with a reagent to generate free hydroxyl or amino sites. These hydrophilic sites are reacted with a protected nucleoside coupling reagent or a linker to protect selected hydroxyl or amino sites. Suitable nucleotide coupling reagents include, for example, a DMT-protected nucleoside phosphoramidite, and DMT-protected H-phosphonate. The unprotected hydroxyl or amino sites is then reacted with a reagent, for example, perfluoroalkanoyl halide, to form hydrophobic sites. The protected hydrophilic sites are then deprotected.

In embodiments of the present teachings, the chemical modality comprises chemical treatment or modification of the surface of the array so as to anchor an amplification reagent to the surface. In some embodiments the amplification reagent is affixed to the surface so as form a patterned array (herein, “immobilized reagent array”) of reaction spots. As referred to herein, “anchor” refers to an attachment of the reagent to the surface, directly or indirectly, so that the reagent is available for reaction during an amplification method of the present teachings, but is not removed or otherwise displaced from the surface prior to amplification during routine handling of the substrate and sample preparation prior to amplification. In some embodiments, the amplification reagent is anchored by covalent or non-covalent bonding directly to the surface of the substrate. In some embodiments, an amplification reagent is bonded, anchored or tethered to a second moiety (“immobilization moiety”) which, in turn, is anchored to the surface of the substrate. In some embodiments of the instant invention, an amplification reagent may be anchored to the surface through a chemically releasable or cleavable site, for example by bonding to an immobilization moiety with a releasable site. The reagent may be released from an array upon reacting with cleaving reagents prior to, during or after the array assembly. Such release methods include a variety of enzymatic, or non-enzymatic means, such as chemical, thermal, or photolytic treatment.

In some embodiments, the amplification reagent comprises a primer, which is released from the surface during a method of the present teachings. In some embodiments, a primer is initially hybridized to a polynucleotide immobilization moiety, and subsequently released by strand separation from the array-immobilized polynucleotides upon array assembly. In another example of primer release, a primers is covalently immobilized on an array via a cleavable site and released before, during, or after array assembly. For example, an immobilization moiety may contain a cleavable site and a primer sequence. The primer sequence may be released via selective cleavage of the cleavable sites before, during, or after assembly. In some embodiments, the immobilization moiety is a polynucleotide which contains one or more cleavable sites and one or more primer polynucleotides. A cleavable site may be introduced in an immobilized moiety during in situ synthesis. Alternatively, the immobilized moieties containing releasable sites may be prepared before they are covalently or noncovalently immobilized on the solid support.

Chemical moieties for immobilization attachment to solid support include those comprising carbamate, ester, amide, thiolester, (N)-functionalized thiourea, functionalized maleimide, amino, disulfide, amide, hydrazone, streptavidin, avidin/biotin, and gold-sulfide groups. Methods of forming immobilized reagent arrays useful herein include methods well known in the art. Such methods are described, for example, in U.S. Pat. No. 5,445,934, Fodor et al., issued Aug. 29, 1995; U.S. Pat. No. 5,700,637, Southern issued Dec. 23, 1997; U.S. Pat. No. 5,700,642, Monforte et al., issued Dec. 23, 1997; U.S. Pat. No. 5,744,305, Fodor et al., issued Apr. 28, 1998; U.S. Pat. No. 5,830,655, Monforte et al., issued Nov. 3, 1998; U.S. Pat. No. 5,837,832, Chee et al., issued Nov. 17, 1998; U.S. Pat. No. 5,858,653, Duran et al., issued Jan. 12, 1999; U.S. Pat. No. 5,919,626, Shi et al., issued Jul. 6, 1999; U.S. Pat. No. 6,030,782, Anderson et al., issued Feb. 29, 2000; U.S. Pat. No. 6,054,270, Southern, issued Apr. 25, 2000; U.S. Pat. No. 6,083,763, Balch, issued Jul. 4, 2000; U.S. Pat. No. 6,090,995, Reich et al., issued Jul. 18, 2000; PCT Patent Publication WO99/58708, Friend et al., published Nov. 18, 1999; Protocols for oligonucleotides and analogs; synthesis and properties, Methods Mol. Biol. Vol. 20 (1993); Beier et al., Nucleic Acids Res. 27: 1970-1977 (1999); Joos et al., Anal. Chem. 247: 96-101 (1997); Guschin et al., Anal. Biochem. 250: 203-211 (1997); Czarnik et al., Accounts Chem. Rev. 29: 112-170 (1996); Combinatorial Chemistry and Molecular Diversity in Drug Discovery, Ed. Kerwin J. F. and Gordon, E. M., John Wiley & Son, New York (1997); Kahn et al., Modern Methods in Carbohydrate Synthesis, Harwood Academic, Amsterdam (1996); Green et al., Curr. Opin. in Chem. Biol. 2: 404-410 (1998); Gerhold et al., TIBS, 24: 168-173 (1999); DeRisi, J., et al., Science 278: 680-686 (1997); Lockhart et al., Nature 405: 827-836 (2000); Roberts et al., Science 287: 873-880 (2000); Hughes et al., Nature Genetics 25: 333-337 (2000); Hughes et al., Cell 102: 109-126 (2000); Duggan, et al., Nature Genetics Supplement 21: 10-14 (1999); and Singh-Gasson et al., Nature Biotechnology 17: 974-978 (1999).

In some embodiments, the immobilization reagent array comprises a hydrogel affixed to the substrate. Hydrogels useful herein include those selected from the group consisting of cellulose gels, such as agarose and derivatized agarose; xanthan gels; synthetic hydrophilic polymers, such as crosslinked polyethylene glycol, polydimethyl acrylamide, polyacrylamide, polyacrylic acid (e.g., cross-linked with dysfunctional monomers or radiation cross-linking), and micellar networks; and mixtures thereof. Derivatized agarose includes agarose which has been chemically modified to alter its chemical or physical properties. Derivatized agarose includes low melting agarose, monoclonal anti-biotin agarose, and streptavidin derivatized agarose. In some embodiments, the hydrogel comprises agarose, derivatized agarose, or mixtures thereof.

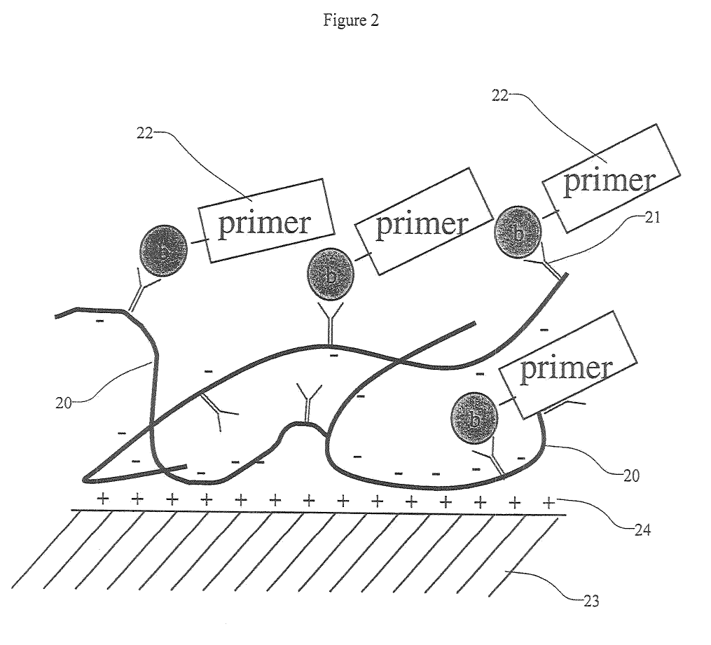

In some embodiments, the substrate comprises a hydrophobic surface. A solution of the hydrogel is then deposited on the surface, such as in a pattern or array, forming reaction spots. Suitable substrates include glass, and plastics selected from the group consisting of polyolefins and polycarbonate. In some embodiments, as depicted in FIG. 2, agarose fibers (20) are mixed with agarose anti-biotin (21) and biotinylated primers (22) or probes (not depicted). The surface of the substrate (23) is treated with APTES or polylysine to make it positively charged (24). The natural negatively charged agarose fibers (20) are held by the positively charged glass (24).

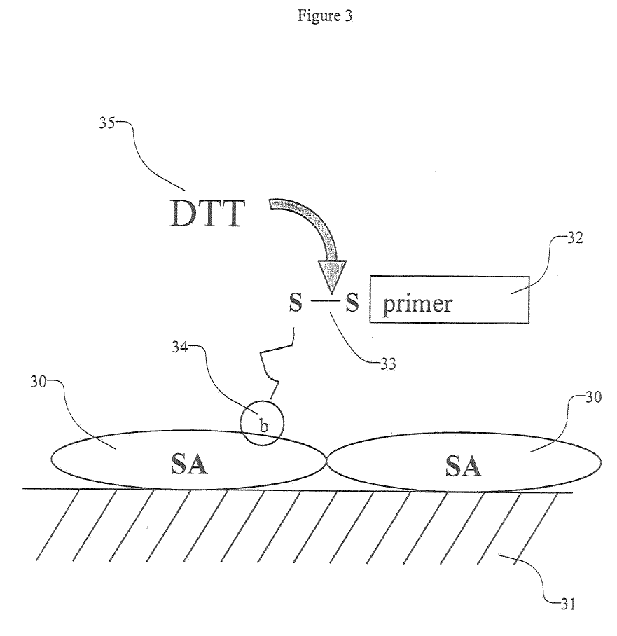

In some embodiments, the immobilized reagent array comprises streptavidin bonded to a substrate. In some embodiments, the substrate is glass. Such methods for binding streptavidin to glass are described, for example, in Birkert, et al., A Streptavidin Surface on Planar Glass Substrates for the Detection of Biomolecular Interaction, 282 Anal. Biochem., 200-208 (2000). In some embodiments, as depicted in FIG. 3, a streptavidin molecule (30) is covalently bonded to the substrate (e.g., glass, 31). An amplification reagent (e.g., a primer, 32) is attached through a disulfide linkage (33) to biotin molecule (34). During a method of the present teachings, the amplification reagent comprises a cleavage reagent (35), such as dithio threitol, to cleave the disulfide linkage, thereby releasing the primer (32) for use in the amplification reaction.

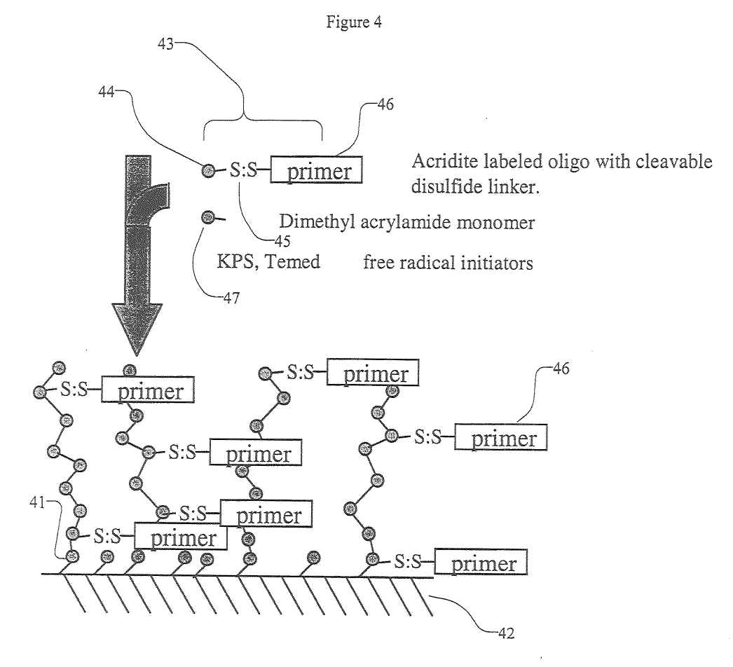

In some embodiments, as depicted in FIG. 4, the immobilization array comprises polacrylamide bonded to a substrate. In this embodiment, an acrylamide monomer (41) is bonded to the surface of the substrate (42). The substrate may comprise glass (such as borosilicate, flint glass, crown glass, float glass), fused silica, and high temperature plastics (such as polycarbonate, polytetrafluoroethylene, poly ether ether ketone, polyamideimide, polypropylene, polydimethyl siloxane). An oligonucleotide (43) is then synthesized with an acridite (44) at the 5′ end, followed by a cleavable linker (45, e.g., disulfide), followed by a primer or probe sequence (46). The acridite labeled oligonucleotide (43) is then polymerized with dimethyl acrylamide monomer (41, 47), in situ, thereby affixing the oligonucleotide to the surface. Methods for immobilizing acrylamid-modified oligonucleotides, among those useful herein, are described in F. Rehman, et al., Immobilization of acrylamide-modified oligonucleotides by co-polymerization, 27 Nucleic Acids Res. 649 (1999). During a method of the present teachings, the amplification reagent comprises a cleavage reagent, such as dithio threitol, to cleave the disulfide linkage, thereby releasing the primer or probe (46) for use in the amplification reaction.

### Sealing Liquid:

The microplates of the present teachings can comprise, during their use, a sealing liquid. As referred to herein, a “sealing liquid” is a material which substantially covers the reaction spots on the substrate of the microplate so as to contain materials present on the reaction spots, and substantially prevent movement of material from one reaction spot to another reaction spot on the substrate. As discussed further herein, the sealing liquid can be coated on the substrate after application of the amplification reagents and liquid sample comprising the polynucleotides to be amplified.

The sealing liquid may be any material which contains the materials on the reaction spots, but is not reactive with those materials under normal storage or amplification conditions. In some embodiments, the sealing liquid is a fluid when it is applied to the surface of the substrate. In some embodiments, the sealing liquid remains fluid throughout the amplification methods of the present teachings. In other embodiments, the sealing liquid becomes a solid or semi-solid after it is applied to the surface of the substrate. In some embodiments, the sealing liquid is substantially immiscible with the amplification reagents and sample of liquid sample.

In some embodiments, the sealing liquid may be transparent, have a refractive index similar to glass, have low or no fluorescence, have a low viscosity, and/or be curable. In some embodiments the sealing liquid comprises a flowable, curable fluid such as a curable adhesive selected from the group consisting of: ultra-violet-curable and other light-curable adhesives; heat, two-part, or moisture activated adhesives; and cyanoacrylate adhesives. Such curable liquids include Norland optical adhesives marketed by Norland Products, Inc. (New Brunswick, N.J., U.S.A.), and cyanoacrylate adhesives, such as disclosed in U.S. Pat. No. 5,328,944, Attarwala et al., issued Jul. 12, 1994; and U.S. Pat. No. 4,866,198, Harris, issued Sep. 12, 1989, and marketed by Loctite Corporation, (Newington, Conn., U.S.A.). In other embodiments, the sealing liquid is selected from the group consisting of mineral oil, silicone oil, fluorinated oils, and other fluids which are substantially non-miscible with water. In some embodiments, the sealing liquid comprises mineral oil.

In some embodiments, the microplates of the present teachings comprise:

(a) a substrate having at least about 10,000 reaction spots, each spot comprising a unique PCR primer and a droplet of PCR reagent having a volume of less than about 20 nanoliters; and

(b) a sealing liquid covering said substrate and isolating each of said reaction spots.

The density of reaction spots (i.e., number of spots per unit surface area of substrate), and the size and volume of reaction spots, may vary depending on the desired application. In some embodiments, the density of the reaction spots on the substrate is from about 10 to about 10,000 spots/cm². In some embodiments, the density of the reaction spots on the substrate is from about 50 to about 1000 spots/cm², such as from about 50 to about 600 spots/cm². In some embodiments, the density is from about 150 to about 170 spots/cm². In some embodiments, the density is from about 480 to about 500 spots/cm². In some embodiments, the area of each site is from about 0.01 to about 0.05 mm². In some embodiments, the area of each site is from about 0.02 to about 0.04 mm². In some embodiments, the volume of the reaction spots is from about 0.05 to about 500 nl, or from about 0.1 to about 200 nl. In some embodiments, the volume is from about 1 to about 5 nl, or about 2 nl. In some embodiments, the volume is less than about 2 nl. In some embodiments, the volume is from about 80 to about 120 nl, or about 100 nl. In some embodiments, the pitch of spots in the array is from about 50 to about 1000 μm, or from about 50 to about 600 μm. In some embodiments, the pitch is from about 400 to 500 μm, or about 450 μm. (As referred to herein, “pitch” is the center-to-center distance between reaction spots.)

In some embodiments, the total number of spots on the substrate is from about 200 to about 100,000, or from about 500 to about 50,000. In some embodiments, the microplate comprises from about 500 to about 10,000 spots, or from about 1,000 to about 7,000 spots. In some embodiments, the microplate comprises from about 10,000 to about 50,000 spots, or from about 15,000 to about 40,000 spots, or from about 20,000 to about 35,000 spots. In some embodiments, the microplate comprises about 30,000 spots.

In some embodiments, the substrate may comprise contain raised or depressed regions, e.g., features such as barriers and trenches to aid in the distribution and flow of liquids on the surface of the substrate. The dimensions of these features are flexible, depending on factors, such as avoidance of air bubbles upon assembly, mechanical convenience and feasibility, etc.

### PCR Equipment:

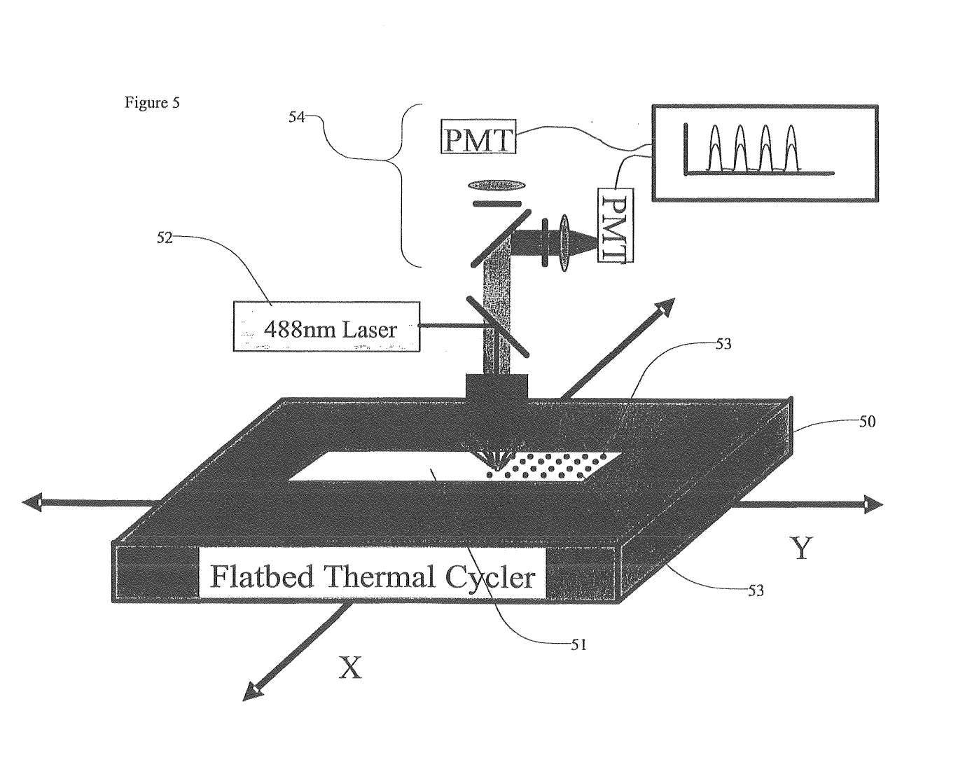

The methods of the present teachings can be performed with equipment which aids in one or more steps of the process, including handling of the microplates, thermal cycling, and imaging. In some embodiments of the present teachings, as generally depicted in FIG. 5, such an amplification apparatus comprises a platform (50) for supporting a microplate (51) of the present teachings, a light source (e.g., laser, 52) for illuminating materials in reaction wells (53), and a detection system (54).

The platform may comprise any device which secures a microplate in the amplification apparatus. In some embodiments, the platform comprises a substantially planar support formed of a material suitable for use in an optical detection system. In some embodiments, the platform is essentially disc-shaped. In some embodiments, the platform is moveable relative to the detection system. Such movement may be by movement of the platform, by movement of the detection system, or both.

In some embodiments, as generally depicted in FIG. 5, the apparatus comprises an optical system which comprises a light source and detection system. In embodiments of the present teachings, the optical system comprises a plurality of lenses, which can be positioned in a linear arrangement; an excitation light source for generating an excitation light; an excitation light direction mechanism for directing the excitation light to a single lens of the plurality of lenses at a time so that a single reaction spot aligned with the well lens is illuminated at a time; and an optical detection system for analyzing light from the reaction spot. The excitation light source directs the excitation light to each of the reaction spots of a row of reaction spots in a sequential manner as the plurality of lenses linearly translates in a first direction relative to the microplate. The plurality of lenses, the microplate, or a combination of the two may be moved, so that a relative motion is imparted between the plurality of lenses and the microplate.

According to some embodiments, the excitation light source provides radiant energy of proper wavelength so as to allow detection of photo-emitting probes in the reaction wells. Depending on the probes used, the light source may emit visible or no-visible wavelengths, including infrared and ultraviolet light. In some embodiments, the excitation source is selected to emit excitation light at one or several wavelengths or wavelength ranges. In some embodiments, the light source comprises a laser emitting light of a wavelength of about 488 nm. In some embodiments, the light source comprises an Argon ion laser. The excitation light from excitation light source may be directed to the reaction spot lenses in any suitable manner. In some embodiments, the excitation light is directed to the lenses by using one or more mirrors to reflect the excitation light at the desired lens. After the excitation light passes through the lens into an aligned reaction spot, the sample in the reaction spot is illuminated, thereby emitting an excitation emission or emitted light. The emitted light can then be detected by an optical system.

In accordance with some embodiments of the present teachings, a detection system is provided for analyzing emission light from the reaction spots. In accordance with some embodiments, the optical system includes a light separating element such as a light dispersing element. Light dispersing elements include elements that separate light into its spectral components, such as transmission gratings, reflective gratings, prisms, and combinations thereof. Other light separating elements include beamsplitters, dichroic filters, and combinations thereof that are used to analyze a single wavelength without spectrally dispersing the incoming light. In embodiments with a single wavelength light processing element, the optical detection device is limited to analyzing a single wavelength, thereby one or more light detectors each having a single detection element may be provided. In some embodiments, the optical detection system may further include a light detection device for analyzing light from a sample for its spectral components. In some embodiments, the light detection device comprises a multi-element photodetector. Multi-element photodetectors include charge-coupled devices (CODs), diode arrays, photo-multiplier tube arrays, charge-injection devices (CIDs), CMOS detectors, and avalanche photodiodes. In some embodiments, the photodetector is a CCD. In some embodiments, the light detection device may be a single element detector. With a single element detector, reaction spots are read one at a time. A single element detector may be used in combination with a filter wheel to take a reading for a single reaction spot at a time. With a filter wheel, the microplate is scanned a large number of times, each time with a different filter. Alternately, other types of single dimensional detectors are one-dimensional line scan CCDs, and single photo-multiplier tubes, where the single dimension could be used for either spatial or spectral separation. It will be understood that alternatively, several single dimension detectors could be used in combination with a dichroic beam splitter

Some embodiments of apparatus useful herein comprise temperature control mechanisms, for example, force convection temperature control mechanisms. Such mechanisms are generally known in the art and include those described in U.S. Pat. No. 5,942,432, Smith et al., issued Aug. 24, 1999; and U.S. Pat. No. 5,928,907, Woundenberg et al., issued Jul. 27, 1999. Temperature control mechanisms may be included to change the temperature of the microplate so as to change the temperature of the samples and reagents placed in the reaction spots. For example, thermal cycling of the sample and reagents may be desirable, particularly in methods of the present teachings for performing PCR or similar amplification reactions.

In some embodiments, such a suitable apparatus comprises a platform for supporting a microplate of the present teachings; a focusing element selectively alignable with an area (e.g., reaction spot) on a microplate; an excitation (light) source to produce an excitation beam that is focused by the focusing element into a selected reaction spot when the focusing element is in the aligned position; and a detection system to detect a selected emitted energy from a sample placed in the reaction well. In embodiments of the present teachings, the focusing element is selectable in an aligned position or an unaligned position relative to at least one of said sample wells. Also, in some embodiments, at least one of said the platform and the focusing element rotates about a selected axis of rotation to move the focusing element between the aligned position and the unaligned position. Apparatus among those useful herein are described, for example, in U.S. Pat. No. 6,015,674, Woudenberg et al., issued Jan. 18, 2000; U.S. Pat. No. 6,563,581, Oldham et al., issued May 13, 2003; and U.S. Patent No. Application Publication 2003/0160957, Oldham et al., published Aug. 28, 2003.

The methods of the present teachings may be performed using commercially available equipment, or modifications thereof so as to accommodate and facilitate the use of the microplates of the present teachings. Such commercially available equipment includes the ABI Prism® 7700 Sequence Detection System, the ABI Prism® 7900 HT instrument, the GeneAmp® 5700 Sequence Detection System, and GeneAmp® PCR System 9600, all of which are marketed by Applied Biosystems, Inc, (Foster City, Calif., U.S.A.).

### Methods

The present teachings provide methods for amplifying a polynucleotide in a liquid sample comprising a plurality of polynucleotide targets, each polynucleotide target being present at very low concentration within the sample. Such methods comprise the steps of applying amplification reactants to the reaction spots; forming a sealed reaction chamber comprising the reaction spots; and subjecting the substrate and reactants to reaction conditions so as to effect amplification. Some embodiments of such methods comprise:

(a) applying amplification reactants to the surface of a substrate comprising reaction spots on the surface of the substrate, wherein the amplification reactants comprise the liquid sample and an amplification reagent mixture;

(b) forming a sealed reaction chamber, having a volume of less than about 20 nanoliters, over each of said reaction spots; and

(c) subjecting the substrate and reactants to reaction conditions so as to effect amplification (e.g., by thermal cycling the substrate and reactants).

In some embodiments, the method comprises performing PCR on a nucleotide in a complex mixture of polynucleotides. In some embodiments, the method comprises simultaneously amplifying a plurality of polynucleotides in a complex mixture of polynucleotides. As referred to herein, “simultaneously amplifying” refers to conducting amplification of two or more polynucleotides in a single mixture of polynucleotides at substantially the same time. In some embodiments, each of the polynucleotides is simultaneously amplified in its own reaction spot.

In some embodiments, the method is conducted on a microplate containing a plurality of reaction spots, wherein each reaction spot comprises reagents for amplifying a single polynucleotide target. In some embodiments, each reaction spot comprises reagents for amplifying one or more targets that are distinct from targets to be amplified in other reaction spots on the microplate. In some embodiments, the microplate comprises a plurality of reaction spots comprising reagents for amplifying the same target or targets.

The major advantage over the prior art provides the benefit of a conservative use of sample. In the prior art case, where a single sample is split amongst many wells and a single analysis is done in each well, most of the sample is put in a well where it will not amplify and will not be detected.

This is a problem in particular for the case of a scarce component in a large number of wells. For instance, if the sample contained ten copies of a given sequence which can only be detected if at least one of these copies winds up in the only well which will amplify and detect it, a method which splits the sample indiscriminately over thousands of wells will not detect it in the vast majority of cases. The only way the prior art can improve this case is to vastly increase the amount of sample used.

The present teachings improve over the prior art because the entire sample, as one pool, is exposed to the microplate surface and allowed time to hybridize to the primers and probes affixed thereon. This process enables the sample to become sorted by sequence onto the spots, which will later become individual reaction volumes. While this process will not have enough time to completely sort out the sample for each and every copy. This enrichment of sequences will increase the probability of detecting rare sequences.

### Polynucleotide Targets:

As referred to herein, a “target” is a polynucleotide comprising nucleotide bases (DNA or RNA) or analogs thereof. In some embodiments, the target comprises at least about 100 bases. Such analogs include peptide nucleic acids (PNA) and locked nucleic acids (LNA). Targets include DNA, such as cDNA (complementary DNA) or genomic DNA, or RNA, such as mRNA (messenger RNA) or rRNA (ribosomal RNA), derived or obtained from any sample or source. In some embodiments, the sample comprising the target is of a scarce or of a limited quantity. For example, the sample may be one or a few cells collected from a crime scene or a small amount of tissue collected via biopsy.

In some embodiments, the target is a chromosome or a gene, or a portion or fragment thereof; a regulatory polynucleotide; a restriction fragment from, for example a plasmid or chromosomal DNA; genomic DNA; mitochondrial DNA; or DNA from a construct or library of constructs (e.g., from a YAC, BAC or PAC library), or RNA (e.g., mRNA, rRNA); or a cDNA or cDNA library. The target polynucleotide may include a single polynucleotide, from which a plurality of different sequences of interest may be amplified, or it may include a plurality of different polynucleotides, from which one or more different sequences of interest may be amplified.

The methods of the present teachings can comprise an amplification of a target from a sample comprising a plurality polynucleotides. In some embodiments, the plurality of polynucleotides comprises a complex mixture of sample polynucleotides. In some embodiments, the complex mixture comprises tens, hundreds, thousands, hundreds of thousands or millions of polynucleotide molecules. In specific embodiments, the amplification methods are used to amplify pluralities of sequences from samples comprising cDNA libraries or total mRNA isolated or derived from biological samples, such as tissues and/or cells, wherein the cDNA, or alternatively mRNA, libraries may be quite large. For example, targets may be amplified from cDNA libraries or mRNA libraries constructed from several organisms, or from several different types of tissues or organs, can be amplified according to the methods described herein. In some embodiments, the complex mixture comprises substantially all of the genetic material from an organism. Such organisms, in some embodiments of the present teachings, include human, mouse, rat, yeast, primate, bacteria, insect, dog, fungus, and virus, including sub-species, strains, and individual subject organisms thereof.

In some embodiments, the present teachings provide methods for the detection of one or more specific targets present in the same or different samples. In some embodiments, the methods also comprise determining the quantity of target in a given sample. Such samples include cellular, viral, or tissue material, such as hair, body fluids or other materials containing genetic DNA or RNA. Embodiments of such methods include those for the diagnosis of disorders, improving the efficiency of cloning DNA or messenger RNA, obtaining large amounts of a desired target from a mixture of nucleic acids resulting from chemical synthesis, and analyzing the expression of genes in a biological system (e.g., in a specific organism, for research or diagnostic purposes). In some embodiments, the present teachings provide methods for analyzing, quantitatively and qualitatively, the expression of the entire genomic material of an organism relative to a known genomic standard. In some embodiments, the present teachings provide methods for simultaneously quantitatively detecting a plurality of polynucleotide targets in a liquid sample comprising a genomic mixture of polynucleotides present at very low concentration, comprising:

(a) distributing the liquid sample into an array of reaction chambers on a planar substrate, wherein
(i) each chamber has a volume of less than about 100 nanoliters, and
(ii) each chamber comprises (1) a PCR primer for one of the polynucleotide targets, and (2) a probe associated with the primer which emits a concentration dependent signal if the PCR primer binds with a polynucleotide, and
(iii) the array comprises at least one chamber comprising a PCR primer for each of the polynucleotide targets;

(b) performing PCR on the samples in the array so as to increase the concentration of polynucleotide in each of the chambers in which the polynucleotide binds to a PCR primer; and

(c) identifying which of the reaction chambers contains a polynucleotide that has been bound to a PCR primer, by detecting the presence of the probe associated with the PCR primer.

The amplification reagent mixture comprises, with reagents that are associated with the reaction spots, the reagents necessary for the amplification reaction to be effected, as discussed above. Such reagents “associated” with reaction spots are those that are contained in or on the reaction spot, as discussed above. In some embodiments, the associated reagents and the amplification reagent mixture comprise distinct reagents (i.e., not having an reagent in common); in other embodiments the associated reagents and the amplification reagent mixture comprise at least one common reagent. In some embodiments, the amplification reaction mixture contains no reagents, and consists essentially of a solvent (e.g., water) in which the sample is dissolved or otherwise mixed. In some embodiments of the present teachings, the associated reagent comprises “target-specific reagents” that are useful in amplifying one or more specific targets. Target specific reagents include such reagents that are specifically designed so as to hybridize to the target or targets, such as primers (or primer pairs) and probes. In some embodiments, the amplification reagent mixture comprises “non-specific reagents” that are regents that are not target specific but are useful in the amplification reaction to be effected. Non-specific reagents include standard monomers for use in constructing the amplicon (e.g., nucleotide triphosphates), polymerases (such as Taq), reverse transcriptases, salts (such as MgCl₂ or MnCl₂), cleavage reagents (such as dithio threitol), and mixtures thereof. In some embodiments of the present teachings, the associated reagents consist essentially of target specific reagents, and the amplification reagent mixture consists essentially of non-specific reagents. In other embodiments, the associated reagents comprise target-specific reagents and non-specific reagents. In other embodiments, the amplification reagent mixture comprises target-specific reagents and non-specific reagents. Reagents among useful herein include those in commercially-available amplification reagent mixtures, including AmpliTaq® Gold PCR Master Mix, TaqMan® Universal Master Mix, and TaqMan® Universal Master Mix No AmpErase® UNG, all of which are marketed by Applied Biosystems, Inc. (Foster City, Calif., U.S.A).

As referred to herein, the “applying” of reactants to the surface of the substrate comprises any method by which the reagents are contacted with the reaction spots in such a manner so as to make the reactants available for amplification reaction(s) in or on the reaction spots. In some embodiments, the reactants are applied in a substantially uniform manner, so that each reaction spot is contacted with a substantially equivalent amount of reagent. As referred to herein, a “substantially equivalent” amount of reagent applied to a reaction spot is an amount which, in combination with the associated reagent, is sufficient to effect amplification of a target in equivalent amounts and timing with other reaction spots on the substrate (consistent with the quantity and nature of targets to be amplified in such reaction spots). In some embodiments, the sample and amplification reaction reagents are mixed prior to application to the surface. In other embodiments, the sample and amplification reagents are applied to the surface separately, either concurrently or sequentially (in either order).

In embodiments of the present teachings, methods of application useful herein include pouring of the reactants onto the surface so as to substantially cover the entire surface (including reaction spots and adjacent areas on the surface). In other embodiments of the present teachings, methods of application comprise spotting or spraying of reactants to specific reaction spots (e.g., by use of pipettes, or automated devices, such as piezoelectric pumps, for delivering microliter quantities of materials). In some embodiments, the application step comprises a dispersion step to effect application of the reactants (or any portion thereof) across the surface of the substrate. Such dispersion methods include use of vacuum, centrifugal force, and combinations thereof. In some embodiments, the sample is applied by pouring the sample on the substrate. In some embodiments, the sample is applied by placing the substrate in a flow cell, wherein the sample is circulated across the surface of the substrate. In some embodiments, the amplification reagent mixture is applied by spraying the reagents onto the surface, wherein the reagents adhere to the hydrophilic reaction spots and do not adhere to adjacent hydrophobic areas on the substrate.

In some embodiments, the application step comprises a reactant removal step, wherein excess reactant is removed after the reactant is applied. In embodiments of the present teachings, the reactant removal step is effected by use of gravity, centrifugal force, vacuum, and combinations thereof. In some embodiments of the present teachings, the reactant removal step is effected using a wiping device, such as a squeegee, which is drawn across the surface of the substrate so as to remove excess reactant. As will be appreciated by one of skill in the art, the wiping device must be contacted to the surface with sufficient force so as to effect removal of excess reactant, without also removing all reactants and associated reagents from the reaction spots. In some embodiments, the application step further comprises an incubation step, after the reactant is applied to the surface but before a reactant removal step (if done), so as to allow the sample to react (e.g., hybridize) with target specific reagents associated with the reaction spots. In some embodiments, the incubation comprises allowing the sample to remain in contact with the surface from about 0.5 to about 50 hours. In embodiments of the present teachings, the application step comprises:

(a) applying the sample;

(b) incubating the sample and associated reagents in the reaction spots; and

(c) applying amplification reagent mixture.

Optionally, the method additionally comprises a reactant removal step after incubating step (b) and before applying step (c). Optionally the method additionally comprises a reactant removal step after applying step (c).

In some embodiments, the targets in the sample are preamplified before the applying step, so as to increase their concentration in the sample. In some embodiments, the methods of the present teachings comprise methods wherein a portion of the sample is preamplified prior to the distributing step, by (1) mixing the portion with reactants comprising a plurality of PCR primers corresponding to the PCR primers in a subset of the chambers of the substrate; (2) thermal cycling the mixture so as to produce a pre-amplified sample; and (3) distributing the preamplified sample to the subset of chambers. In some embodiments, the plurality of PCR primers comprises from about 100 to about 1000 primer sets. In some embodiments, the plurality of primers comprises from about 2 to about 50 primer sets.

The forming of the reaction chambers is effected by any method by which the contents of each reaction spot are physically isolated from adjacent reaction spots. As referred to herein, “physical isolation” refers to the creation of a barrier which substantially prevents physical transfer of reactants or amplification reaction products (e.g., amplicons) between reaction chambers. In some embodiments, such method of physical isolation also physically isolates the reaction chambers from the environment, such that reactants and reaction products are not lost to the air or to surrounding surfaces of the microplate through, e.g., evaporation. In some methods, the forming of the reaction chamber is effected by applying a sealing fluid to the surface of the substrate. Such methods of applying include those described above regarding the application of reactants.

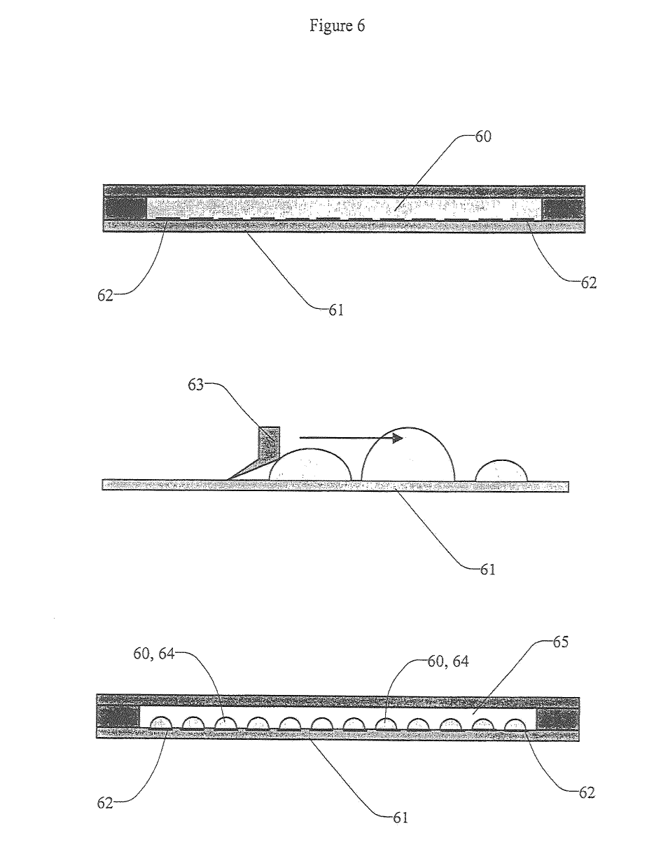

One embodiment of the present teachings is depicted in FIG. 6, wherein a sample (60) is applied to the surface of a substrate (61) which comprises a plurality of reaction spots (62). The excess sample is then removed from the surface using a squeegee (63). Amplification reagent mixture (64) is then applied to the surface, followed by application of a sealing fluid (65) which coats the surface of the substrate, including the reaction spots. The substrate and reactants are then subjected to thermal cycling to effect amplification of targets in the sample.

### Kits

The present teachings also provide reagents and kits suitable for carrying out polynucleotide amplification. Such reagents and kits may be modeled after reagents and kits suitable for carrying out conventional PCR, RT-PCR, and other amplification reactions. Such kits comprise a microplate of the present teachings and a reagent selected from the group consisting of an amplification reagent, a detection reagent, and combinations thereof. Examples of specific reagents include, but are not limited, to the reagents present in AmpliTaq® Gold PCR Master Mix, TaqMan® Universal Master Mix, and TaqMan® Universal Master Mix No AmpErase® UNG, Assays-by-Design^{SM}, Pre-Developed Assay Reagents (PDAR) for gene expression, PDAR for allelic discrimination. and Assays-On-Demand®, all of which are marketed by Applied Biosystems, Inc. (Foster City, Calif., U.S.A.). The kits may comprise reagents packaged for downstream or subsequent analysis of the multiplex amplification product. In some embodiments, the kit comprises a container comprising a plurality of amplification primer pairs or sets, each of which is suitable for amplifying a different sequence of interest, and a plurality of reaction vessels, each of which includes a single set of amplification primers suitable for amplifying a sequence of interest The primers included in the individual reaction vessels can, independently of one another, be the same or different as a set of primers comprising the plurality of multiplex amplification primers.

The materials, devices, apparatus and methods of the present teachings are illustrated by the following non-limiting Examples.

### Example 1

An amplification method of the present teachings is performed using a surface-treated microscope slide, supplied by Scienion AG (Berlin, Germany), on which discrete hydrophilic areas are created. Each spot is essentially circular in shape, having a diameter of about 160 μm. An array of 30,000 spots is formed on the surface of the slide. Sets of PCR primers and probes, for hybridizing with known oligonucleotides, are then deposited on the hydrophilic areas and covalently linked to the hydrophilic surface through a cleavable disulfide linker, forming reaction spots. A unique set of primers and probes is deposed on each spot.

A sample containing a mixture of polynucleotides is then flooded across the surface of the slide, contacting the reaction spots. The sample is allowed to incubate for about twelve hours, after which excess sample is removed from the surface using a squeegee. An amplification reagent mixture comprising a disulfide cleavage agent (TaqMan® Universal Master Mix, marketed by Applied Biosystems, Inc., Foster City, Calif., U.S.A, modified to comprise an elevated amount of dithio threitol) is then sprayed onto the surface of the slide, adhering to the reaction spots. (The dithio threitol cleaves the disulfide linkage of the covalently attached probes and primers, thereby releasing the primers and probes for the amplification reaction.)

The volume of PCR reactants in each reaction spot is less than 2 nl. The surface is then flooded with mineral oil, and the slide placed in a ABI Prism® 7900 HT instrument, which is modified to illuminate and scan finely-spaced reaction spots. The substrate and PCR reactants are then thermally cycled, The number of cycles is then determined for amplicons to be produced in each reaction spot reaching detection levels, thereby allowing qualitative and quantitative analysis of oligonucleotides in the sample according to conventional analytical methods.

### Example 2

A microplate is made according to the present teachings, by applying discrete spots of agarose onto a polycarbonate plastic substrate. A solution is made comprising 3% (by weight) of agarose having a melt point≦65° C., supplied as NuSieve GTG, by FMC BioProducts (Rocland, Me., U.S.A). The solution is then spotted onto the surface of the substrate in an array comprising 15,000 reaction spots. The microplate is then used in a method according to Example 1. In this method, High Resolution blend Agarose 3:1, and Monoclonal anti-biotin-agarose, supplied by Sigma (St. Louis, Mo., U.S.A) are substituted for the low melt agarose, with substantially similar results.

### Example 3

A microplate is made according to the present teachings, by cutting an optical adhesive cover, P/N 4311971, supplied by Applied Biosystems Inc. (Foster City, Calif., U.S.A) to the size of a standard glass slide, and pasting the cover to the slide. Heat and pressure is applied while smoothing the cover over the glass surface in order to expel air bubbles between the cover and glass surface. 2 uL droplets of 1% low melting agarose are delivered onto the plastic surface at a 450 μm pitch in a matrix and dried at low heat on a hot plate. The plastic surface is rinsed with deionized water. A matrix of water droplets is retained on the plastic surface when the excess of water was removed. 2 uL of RNase P TaqMan® reaction mix, supplied by Applied Biosystems, Inc. (Foster City, Calif., U.S.A) with human genomic DNA is then added onto each spot and covered with mineral oil. Thermal cycling and fluorescence detection are then carried out using a PCR instrument that is compatible with glass slides.

The examples and other embodiments described herein are exemplary and not intended to be limiting in describing the full scope of compositions and methods of the present teachings. Equivalent changes, modifications and variations of specific embodiments, materials, compositions and methods may be made within the scope of the present teachings, with substantially similar results.

What is claimed is:

1. A method for performing PCR on a liquid sample comprising a plurality of polynucleotide targets, each polynucleotide target being present at very low concentration within the sample, comprising:
applying PCR reactants to the surface of a substrate to produce a plurality of reaction spots on the surface of the substrate;
loading the liquid sample and a PCR reagent mixture onto the reaction spots;
forming a sealed reaction chamber, having a volume of less than about 20 nanoliters, over each of the reaction spots; and
amplifying the sample.

2. A method according to claim 1, wherein said surface of the substrate comprises a plurality of reaction spots, wherein each spot comprises PCR reactants comprising at least one probe and set of primers for one or more targets among said polynucleotide targets.

3. A method according to claim 1 further comprising loading said liquid sample and said reagent mixtures in separate steps.

4. A method according to claim 3 further comprising removing said liquid sample from said surface prior to said applying of said PCR reagent mixture.

5. A method according to claim 3, comprising the additional sub-step of removing said PCR reagent mixture from the surface of said substrate adjacent to said reaction spots, after applying of said PCR reagent mixture.

6. A method according to claim 1, wherein the applying said PCR reactants comprises spraying said reactants on said surface of the substrate.

7. A method according to claim 1, wherein said forming comprises loading a sealing fluid on said surface of the substrate so as to substantially cover the reaction spots.

8. A method according to claim 1, wherein said reaction chamber has a volume of from about 1 to about 5 nanoliters.

9. A method according to claim 1 further comprising providing said substrate comprising hydrophobic regions and hydrophilic reaction spots.

10. A method according to claim 1 further comprising depositing a hydrophilic material to said reaction spots on said substrate before the applying PCR reactants.

11. A method according to claim 1 further comprising producing at least about 10,000 reaction spots.

12. A method according to claim 1 further comprising detecting an amplification of the sample.

13. A method for simultaneously quantitatively detecting a plurality of polynucleotide targets in a liquid sample comprising a genomic mixture of polynucleotides present at very low concentration, comprising:
(a) distributing the liquid sample into an array of reaction chambers on a planar substrate, wherein
(i) each chamber has a volume of less than about 100 nanoliters, and
(ii) each chamber comprises (1) at least one amplification primer for one of the polynucleotide targets, and (2) a probe associated with the primer which emits a concentration dependent signal if the amplification primer binds with a polynucleotide, and
(iii) the array comprises at least one chamber comprising at least one amplification primer for each of the polynucleotide targets;
(b) performing amplification on the samples in the array so as to increase the concentration of polynucleotide in each of the chambers in which the polynucleotide binds to a amplification primer; and
(c) identifying which of the reaction chambers contains a polynucleotide that has been bound to a amplification primer, by detecting the presence of the probe associated with the amplification primer.

14. A method according to claim 13 further comprising preamplifying the sample prior to the distributing step, by (1) mixing the portion with reactants comprising a plurality of amplification primers corresponding to the amplification primers in a subset of the chambers of the substrate; (2) thermal cycling the mixture so as to produce a pre-amplified sample; and (3) distributing the preamplified sample to the subset of chambers.

15. A method according to claim 13 further comprising affixing an amplification reagent to each reaction spot of said surface of said substrate.

16. A method according to claim 14, wherein said surface of the substrate comprises a plurality of reaction spots, wherein each spot comprises at least one probe and at least one set of primers for one or more targets among said polynucleotide targets.

17. A method according to claim 13 further comprising loading said liquid sample and said reagent mixtures in separate steps.

18. A method according to claim 17 further comprising removing said liquid sample from said surface prior to said applying of said PCR reagent mixture.

19. A method according to claim 17, comprising the additional sub-step of removing said PCR reagent mixture from the surface of said substrate adjacent to said reaction spots, after applying of said PCR reagent mixture.

20. A method according to claim 13 further comprising loading a sealing fluid on said surface of the substrate so as to substantially cover the reaction spots.
